(19) United States
(12) Patent Application Publication
(10) Pub. No.: US 2025/0057890 A1
(43) Pub. Date: Feb. 20, 2025

(54) FUNCTIONAL FOOD CONTAINING BONITO-DERIVED EXTRACT FOR PREVENTING OR AMELIORATING NEUROPSYCHIATRIC DISORDERS

(71) Applicants: MARUHACHI MURAMATSU, INC., Shizuoka (JP); NIPPON MEDICAL SCHOOL FOUNDATION, Tokyo (JP)

(72) Inventors: Yoshinori HOKARI, Shizuoka (JP); Aya SEKINE, Shizuoka (JP); Yoshihiko KAKINUMA, Tokyo (JP)

(21) Appl. No.: 18/726,183

(22) PCT Filed: Jan. 10, 2023
(86) PCT No.: PCT/JP2023/000272

§ 371 Date: Jul. 2, 2024

Related U.S. Application Data
(60) Provisional application No. 63/266,531, filed on Jan. 7, 2022.

(51) Int. Cl. A61K 35/60; A61P 25/00; A61P 29/00

(52) U.S. Cl. CPC A61K 35/60; A61P 25/00; A61P 29/00

(57) ABSTRACT

The present invention is directed to the provision of a functional food that has use in reasonably preventing or ameliorating neuropsychiatric disorders. In accordance with the present invention, there is provided a functional food for preventing or ameliorating a neuropsychiatric disorder, the functional food being characterized by comprising bonito-derived extract obtained by hot-water or cold-water extraction of katsuo-arabushi. The neuropsychiatric disorder is caused by inflammation of the brain or breakdown of the blood-brain barrier, and the functional food has anti-inflammatory action, blood-brain barrier property-improving action, and cardiac acetylcholine production system-activating action.

### FIELD OF THE INVENTION

The present invention relates to a functional food containing bonito-derived extract for preventing or ameliorating neuropsychiatric disorders.

### BACKGROUND OF THE INVENTION

With the increase in the elderly population, the number of patients suffering from neuropsychiatric disorders such as Alzheimer's disease, Parkinson's disease, and schizophrenia is on the rise; however, no radical therapy has yet been established for any of these conditions, and the discovery of methods for preventing or delaying the onset thereof is a pressing issue.

In view of this societal background, it has gradually become apparent in recent years that food ingredients affect brain function, and there is growing interest in the preventive effects of daily intake of functional foods.

### PRIOR ART LITERATURE

### Patent Literature

[Patent Document 1] JP 2017-008104
[Patent Document 2] JP 2015-093845

### BRIEF SUMMARY OF THE INVENTION

### Issues to be Solved by the Invention

The present invention was developed in view of the circumstances described above, and is directed to the provision of a functional food that has use in reasonably preventing or ameliorating neuropsychiatric disorders.

### Means to Solve Issues

The inventors made the following discoveries about functional foods that prevent or improve neuropsychiatric disorders, and, after diligent experimentation and other efforts, arrived at the present invention.

Specifically, it has been reported in recent years that dysfunction in the blood-brain barrier (BBB) are involved in neuropsychiatric disorders such as Alzheimer's disease, Parkinson's disease, and schizophrenia. The blood-brain barrier plays an important role in keeping the cerebral environment constant by isolating the brain from substances flowing into the brain parenchyma in the blood and controlling the circulation of substances inside and outside the brain. However, when the function thereof breaks down, there is direct contact between neurons and substances and the like that are harmful to the brain, which is said to cause neurological cell death and a decline in neurological activity accompanied by inflammation of the brain.

The inventors found that the cardiomyocytes of the heart have their own system (the non-neuronal, non-central cardiac acetylcholine production system; NNCCS) for producing acetylcholine (ACh). It also came to light that the physiological function of the cardiomyocyte NNCCS modifies the functioning of the central nervous system, in addition to the circulatory system, via the vagus nerve. For example, a novel function was discovered: the NNCCS is involved in maintaining the blood-brain barrier (BBB), and bears inter-organ crosstalk through this system. As of yet, no system for indirectly enhancing BBB function via the nerves has been reported, and this is a specific mode that is considered very highly novel. It has also been suggested that the activation of this system may lead to the enhancement of cardiovascular function and the prevention of disease, and candidate substances for the enhancement of NNCCS function have currently been identified.

Focusing on bonito-derived extract, the inventors discovered novel health functions thereof in the form of anti-inflammatory action, improved blood-brain barrier properties, and activation of the NNCCS and, informed by these discoveries, engaged in diligent experimentation and other efforts regarding the efficacy thereof as a functional food, thereby arriving at the present invention.

Specifically, the present invention provides the following.
(1) A functional food for preventing or ameliorating a neuropsychiatric disorder, the functional food being characterized by comprising bonito-derived extract.
(2) The functional food according to (1), wherein the neuropsychiatric disorder is caused by inflammation of the brain.
(3) The functional food according to (2), wherein the functional food has anti-inflammatory action.
(4) The functional food according to (3), wherein the anti-inflammatory action is inhibition of inflammatory cytokine production and/or inhibition of microglial activation in the brain.
(5) The functional food according to (3), wherein the bonito-derived extract reduces the production of inflammatory cytokines compared to the amounts of DHA and EPA in the bonito-derived extract and a composition containing comparable concentrations of DHA and EPA.
(6) The functional food according to (3), wherein the bonito-derived extract reduces the production of inflammatory cytokines compared to the amounts of histidine, anserine, creatine, creatinine, betaine, carnosine, and inosinic acid in the bonito-derived extract and a composition containing comparable concentrations of histidine, anserine, creatine, creatinine, betaine, and carnosine.
(7) The functional food according to (1), wherein the neuropsychiatric disorder is caused by breakdown of the blood-brain barrier.
(8) The functional food according to (7), wherein the functional food has an effect of improving blood-brain barrier properties.
(9) The functional food according to (8), wherein the active ingredients in the effect of improving blood-brain barrier properties are histidine and inosinic acid.
(10) The functional food according to (7), wherein the functional food has an effect of activating the NNCCS.
(11) The functional food according to (1), wherein the concentration of the bonito-derived extract is 0.1 mg/mL.
(12) The functional food according to (9), wherein the concentration of the bonito-derived extract is 0.1 mg/ml, or the concentration of histidine is 0.836 mg/ml and the concentration of inosinic acid is 0.0537 mg/mL.
(13) The functional food according to (10), wherein the concentration of the bonito-derived extract is 10 mg/mL.

By virtue of the features described above, the functional food characterized by containing bonito-derived extract according to the present invention is capable of exhibiting the effects of anti-inflammatory action, improvement of blood-brain barrier properties, and activation of the NNCCS.

Characteristics of the present invention other than those described above will become apparent from the description of embodiments of the present invention hereinafter.

### BRIEF DESCRIPTION OF THE DRAWINGS

FIG. 1 is a schematic diagram of an experiment to evaluate the anti-inflammatory action of bonito-derived extract and the like using MG6 cells.

FIG. 2 shows experimental results for an evaluation of the anti-inflammatory action of katsuo-arabushi (smoked, dried bonito fillets) (hot water extract and cold water extract).

FIG. 3 shows experimental results for an evaluation of the anti-inflammatory action of katsuo-honkarebushi (smoked, sun-dried, and fermented bonito fillets) (hot water extract and cold water extract).

FIG. 4 shows experimental results for an evaluation of the anti-inflammatory action of namaribushi (smoked bonito fillets) (hot water extract).

FIG. 5 shows experimental results for an evaluation of the anti-inflammatory action of urumebushi (smoked, dried round herring) and sababushi (smoked, dried mackerel fillets) (hot water extract and cold water extract).

FIG. 6 shows experimental results for an evaluation of the anti-inflammatory action of sōdabushi (smoked, dried frigate tuna fillets) and magurobushi (smoked, dried yellowfin tuna fillets) (hot water extract and cold water extract).

FIG. 7A shows results for cerebral expression of inflammatory cytokine genes and blood corticosterone concentration in an in vivo experiment (katsuo-arabushi hot water extract) using inflammation-induced mice under a restraint stress load.

FIG. 7B shows results for microglial activity in the hypothalamus in an in vivo experiment (katsuo-arabushi hot water extract) using inflammation-induced mice under a restraint stress load.

FIG. 8A shows results for hepatic expression of inflammatory cytokine genes in an in vivo experiment (katsuo-arabushi hot water extract) using mice with LPS-induced inflammation.

FIG. 8B shows results for hepatic expression of α7 nicotinic receptor protein in an in vivo experiment (katsuo-arabushi hot water extract) using mice with LPS-induced inflammation.

FIG. 8C shows results for inflammatory cytokine levels in the blood in an in vivo experiment (katsuo-arabushi hot water extract) using mice with LPS-induced inflammation.

FIG. 9 shows experimental results for an evaluation, using MG6 cells, of the anti-inflammatory action of fractions obtained by separating and purifying katsuo-arabushi hot water extract via gel-filtration chromatography.

FIG. 10 shows experimental results for an evaluation, using MG6 cells, of the anti-inflammatory action of fractions obtained by further separating and purifying a gel-filtration active fraction I via reversed-phase HPLC.

FIG. 11 shows experimental results for an evaluation, using MG6 cells, of the anti-inflammatory action of fractions obtained by further separating and purifying a gel-filtration active fraction II via reversed-phase HPLC.

FIG. 12 shows experimental results for an evaluation of the anti-inflammatory action of components (creatinine, glycolic acid, and lactic acid) detected in quantities from a fraction 7 of the gel-filtration active fraction II.

FIG. 13 shows experimental results for an evaluation of the anti-inflammatory action of components (inosinic acid, AMP [adenosine 5′-monophosphate], succinic acid, ribose 5-phosphate, and hypoxanthine) detected in quantities from a fraction 17 of the gel-filtration active fraction II.

FIG. 14 shows experimental results for an evaluation, using MG6 cells, of the anti-inflammatory action of fractions obtained by further separating and purifying a gel-filtration active fraction III via reversed-phase HPLC.

FIG. 15 shows experimental results for an evaluation, using rat brain capillary endothelial cells, of the effects of katsuo-arabushi hot water extract on protein expression of tight-junction-associated molecules.

FIG. 16 shows experimental results for an evaluation, using mouse brains, of the effects of katsuo-arabushi hot water extract on protein expression of acetylcholine synthase (ChaT; choline acetyl transferase).

FIG. 17 shows experimental results for an evaluation, using rat brain capillary endothelial cells, of the effects of a highly-active anti-inflammatory fraction obtained by successively separating and purifying katsuo-arabushi hot water extract using gel-filtration chromatography and reversed-phase HPLC upon protein expression of tight-junction-associated molecules.

FIG. 18 shows experimental results for an evaluation, using rat brain capillary endothelial cells, of the effects of components (inosine and histidine) expected to be present in a highly-active anti-inflammatory fraction and of Dashi-presso (boxed katsuo-dashi [bonito soup stock] produced by Maruhachi Muramatsu, Inc.) upon protein expression of tight-junction-associated molecules.

FIG. 19 shows results for improvement in blood-brain barrier function in an in vivo experiment (katsuo-arabushi hot water extract) using cryoinjured mice.

FIG. 20A shows experimental results for an evaluation of the effects of katsuo-arabushi hot water extract upon cardiac acetylcholine production capacity in mice in terms of tissue acetylcholine level.

FIG. 20B shows experimental results for an evaluation of the effects of katsuo-arabushi hot water extract upon hemodynamic changes in mice.

FIG. 21 shows experimental results for a forced swim test using mice receiving oral katsuo-arabushi hot water extract.

FIG. 22 shows experimental results for a tail suspension test using mice receiving oral katsuo-arabushi hot water extract.

FIG. 23 is a schematic diagram of a novel object recognition test to evaluate visual cognitive memory in mice.

FIG. 24 shows experimental results for a novel object recognition test using mice receiving oral katsuo-arabushi hot water extract.

### DETAILED DESCRIPTION OF THE INVENTION

An embodiment of the present invention will now be described with reference to the drawings, etc.

### Embodiment of the Present Invention

First of all, the sequence of events by which the conclusion that bonito-derived extract has the effects of anti-inflammatory action, improving blood-brain barrier properties, and activating the non-neuronal, non-central cardiac acetylcholine production system was reached will be explained.

### (Anti-Inflammatory Action)

The anti-inflammatory action of extracts from katsuobushi (smoked, dried, and optionally fermented bonito, including arabushi, honkarebushi, and namaribushi preparations), which is a traditional Japanese food product, as well as smoked fish extracts from various other types of fish (round herring, mackerel, frigate tuna, and yellowfin tuna) was screened with an in vitro experimental system using a cultured cell line from mouse brain microglia. As a result, anti-inflammatory action was observed in almost all of the smoked fish extracts; however, the anti-inflammatory action of DHA and EPA, which are omega-3 polyunsaturated fatty acids unique to fish, is already known, and it was believed that the anti-inflammatory action of the various smoked fish extracts might be due to these fatty acids.

Thus, the DHA and EPA content levels of the various smoked fish extracts were calculated via GCMS analysis, compositions having levels thereof comparable to the various extracts were prepared using DHA and EPA reagents with reference to the calculated levels, and the anti-inflammatory action thereof was investigated, whereupon it was revealed that the quantities of DHA and EPA in the katsuobushi extracts were extremely low for anti-inflammatory action to be observed and that the katsuo-arabushi extract had higher anti-inflammatory effects than the composition having comparable levels of DHA and EPA, and it was hypothesized that a component having high anti-inflammatory effects other than DHA or EPA was present in the katsuobushi extracts. Meanwhile, most of the other smoked fish extracts contained more DHA and EPA than the katsuobushi extract, and the various extracts had anti-inflammatory effects comparable to that of the compositions having comparable levels of DHA and EPA; thus, it was believed that DHA and EPA were the responsible substances. In the light of these circumstances, it was decided to use katsuo-arabushi as the research material.

In addition, the anti-inflammatory action of known components characteristic of katsuobushi (histidine, anserine, creatine, creatinine, betaine, carnosine, and inosinic acid) was investigated using the same evaluation system; however, none of these substances in isolation showed anti-inflammatory action comparable to katsuobushi extract, and no pronounced anti-inflammatory action was observed even when these components (except for inosinic acid) were mixed. From these facts, it was believed that there was an unknown substance present in katsuobushi extract that exhibited strong anti-inflammatory action.

### (Blood-Brain Barrier Properties-Improving Action)

Using the anti-inflammatory action of cultured cells from mouse brain microglia as a benchmark, the expression of tight-junction-associated molecules (claudin-5 and occludin) in rat cerebrovascular endothelial cells was investigated for active fractions obtained during the process of sequentially separating and purifying the katsuo-arabushi hot water extract via gel-filtration chromatography and reversed-phase HPLC to identify the active ingredients therein, as well as compounds (histidine and inosinic acid) estimated from the active fractions via LCMS analysis; elevated expression was observed at both the genetic level and the protein level. These effects were observed even when the mice received katsuo-arabushi hot water extract or an active fraction orally. In addition, BBB breakdown was significantly inhibited in mice receiving oral katsuo-arabushi hot water extract in a cryoinjury model of BBB breakdown.

### (Activation of the NNCCS)

As a result of oral administration of katsuo-arabushi hot water extract to mice, cardiac acetylcholine levels increased, and elevated expression of acetylcholine synthase (ChAT) at the protein level was also observed in the heart and brain, confirming that the cardiac acetylcholine production system was activated. In addition, there was a significant reduction in heart rate in mice receiving oral katsuo-arabushi hot water extract, from which it was confirmed that there was, simultaneously, a systemic elevation in parasympathetic nervous system activity.

Below are descriptions of the experiments performed to investigate whether bonito-derived extract has the effects of anti-inflammatory action, improving blood-brain barrier properties, and activating the NNCCS, as well as the results of said experiments.

### [Experiment 1]

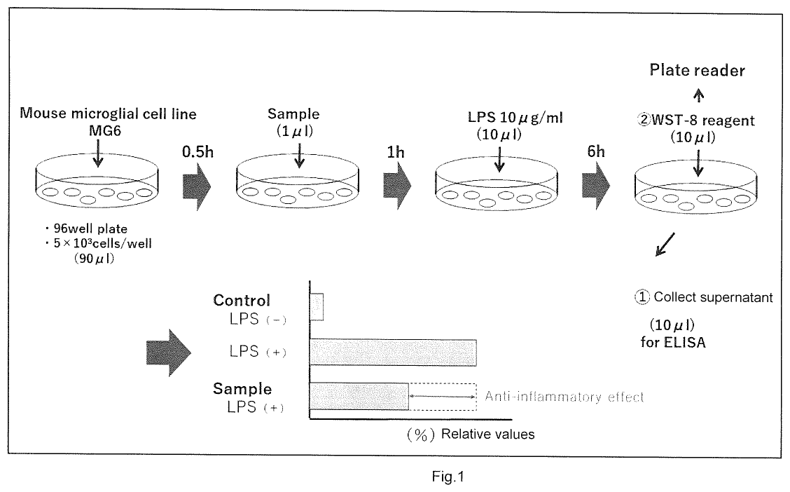

MG6 cells from mouse brain microglia were purchased from the RIKEN BioResource Research Center (BRC), and the anti-inflammatory action of various extracts, including from katsuobushi, were assayed (see FIG. 1). First, a 96-well plate was seeded with the MG6 cells (5×10³ cells/well·90 μL), and a culture was started in a CO₂ incubator (37° C., CO₂ level 5%). Next the various extracts were added to the wells after 0.5 hours, followed by the addition of lipopolysaccharide (LPS) 1 hour after that to activate the MG6 cells and induce an inflammatory state. After 6 hours, 10 μL of culture supernatant was collected from each well, after which 10 μL of a viable cell counting reagent (WST-8) was added to each well, and absorbance at 450 nm (reference wavelength 630 nm) was measured with a plate reader after 1 hour and 2 hours.

The previously collected culture supernatant was diluted 25-fold with a buffer, and the levels of TNF-α, a type of inflammatory cytokine, produced in the culture supernatant was measured via ELISA (using ELISA MAX™ Deluxe Set Mouth TNF-α, a kit manufactured by BioLegend). In the LPS addition test section, anti-inflammatory action was determined to be higher when the TNF-α production levels were more reduced through the prior addition of the various extracts, and lower the less said levels were reduced.

### [Experiment 2]

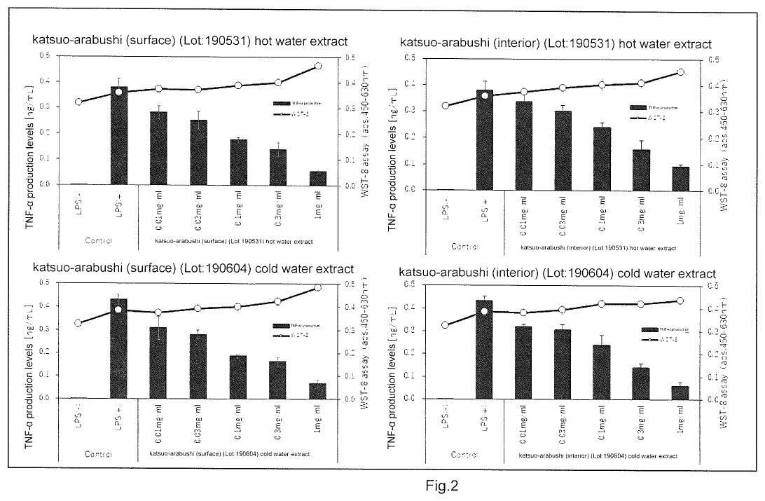

The anti-inflammatory action of katsuo-arabushi hot water extract and cold water extract (0.01 mg, 0.03 mg, 0.1 mg, 0.3 mg, and 1 mg/mL) against LPS stimulation was evaluated (see FIG. 2). From the fact that TNF-α production levels decreased in a concentration-dependent manner regardless of the part (surface or interior) of the katsuo-arabushi or the extraction method used, it was revealed that there was anti-inflammatory action. In the WST-8 assay, no reduction in absorption values in the test sections for the various extracts compared to the control test section (LPS±) were observed, from which it could be confirmed that anti-inflammatory action was exhibited without affecting cell viability.

Furthermore, the DHA and EPA content levels of the various extracts were calculated via GCMS analysis, compositions having levels thereof comparable to the various extracts were prepared using DHA and EPA reagents with reference to the calculated levels, and the anti-inflammatory action thereof was investigated, whereupon it was revealed that the quantities of DHA and EPA in the katsuo-arabushi extracts were extremely low for anti-inflammatory action to be observed, and the katsuo-arabushi extracts also had higher anti-inflammatory effects than the compositions having comparable levels of DHA and EPA; thus, it was hypothesized that another component exhibiting high anti-inflammatory action other than DHA or EPA was present.

### [Experiment 3]

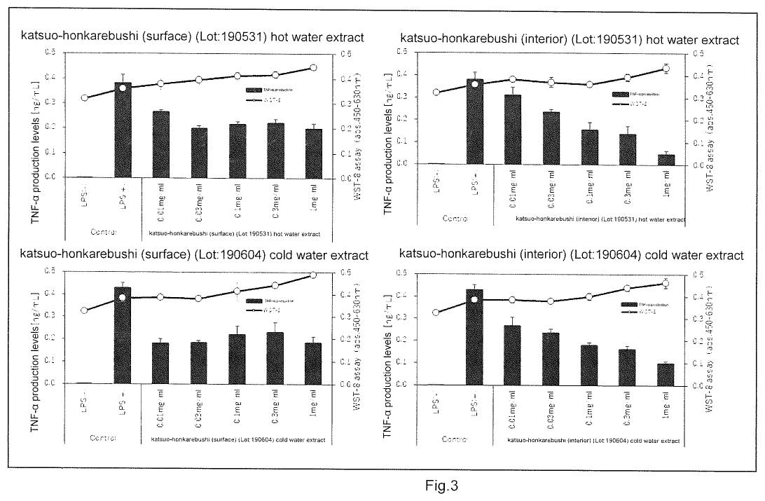

The anti-inflammatory action of katsuo-honkarebushi hot water extract and cold water extract (0.01 mg, 0.03 mg, 0.1 mg, 0.3 mg, and 1 mg/mL) against LPS stimulation was evaluated (see FIG. 3). From the fact that TNF-α production levels decreased in a concentration-dependent manner regardless of the part (surface or interior) of the katsuo-honkarebushi or the extraction method used, it was revealed that there was anti-inflammatory action. In the WST-8 assay, no reduction in absorption values in the test sections for the various extracts compared to the control test section (LPS±) were observed, from which it could be confirmed that anti-inflammatory action was exhibited without affecting cell viability.

Furthermore, when the DHA and EPA content levels of the various extracts were calculated via GCMS analysis, the levels of DHA and EPA in the katsuo-honkarebushi extracts were extremely low for anti-inflammatory effects to be observed; thus, it was hypothesized that another component exhibiting high anti-inflammatory other than DHA or EPA was present.

### [Experiment 4]

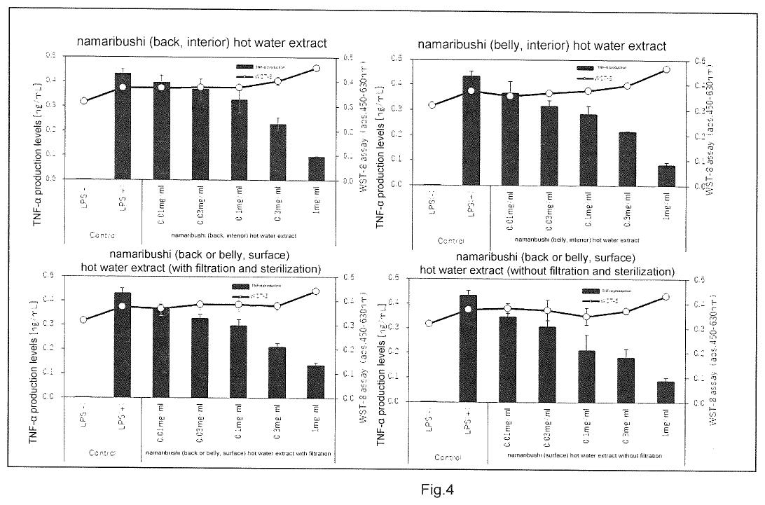

The anti-inflammatory action of namaribushi hot water extract (0.01 mg, 0.03 mg, 0.1 mg, 0.3 mg, and 1 mg/mL) against LPS stimulation was evaluated (see FIG. 4). From the fact that TNF-α production levels decreased in a concentration-dependent manner regardless of the part (back or belly, surface or interior) of the namaribushi or the extraction method used, it was revealed that there was anti-inflammatory action. In the WST-8 assay, no reduction in absorption values in the test sections for the various extracts compared to the control test section (LPS±) were observed, from which it could be confirmed that anti-inflammatory action was exhibited without affecting cell viability.

Furthermore, when the DHA and EPA content levels of the various namaribushi extracts were calculated via GCMS analysis, the levels of DHA and EPA in the namaribushi extracts were extremely low for anti-inflammatory effects to be observed; thus, it was hypothesized that another component exhibiting high anti-inflammatory other than DHA or EPA was present.

### [Experiment 5]

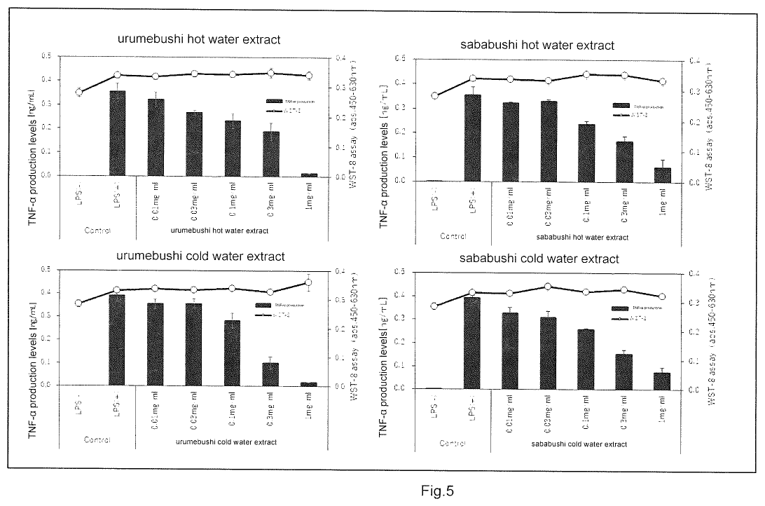

The anti-inflammatory action of hot water extracts and cold water extracts of urumebushi and sababushi (0.01 mg, 0.03 mg, 0.1 mg, 0.3 mg, and 1 mg/mL) against LPS stimulation was evaluated (see FIG. 5). From the fact that TNF-α production levels decreased in a concentration-dependent manner regardless of the extraction method used on the urumebushi or sababushi, it was revealed that there was anti-inflammatory action. In the WST-8 assay, no reduction in absorption values in the test sections for the various extracts compared to the control test section (LPS±) were observed, from which it could be confirmed that anti-inflammatory action was exhibited without affecting cell viability.

However, when the DHA and EPA content levels of the various extracts were calculated via GCMS analysis, compositions having levels thereof comparable to the extracts were prepared using DHA and EPA reagents, and the anti-inflammatory action thereof was investigated, the anti-inflammatory effects of the various extracts and the compositions containing comparable levels of DHA and EPA were comparable; thus, it was considered highly likely that the substances responsible for the anti-inflammatory action observed in the various extracts were DHA and EPA.

### [Experiment 6]

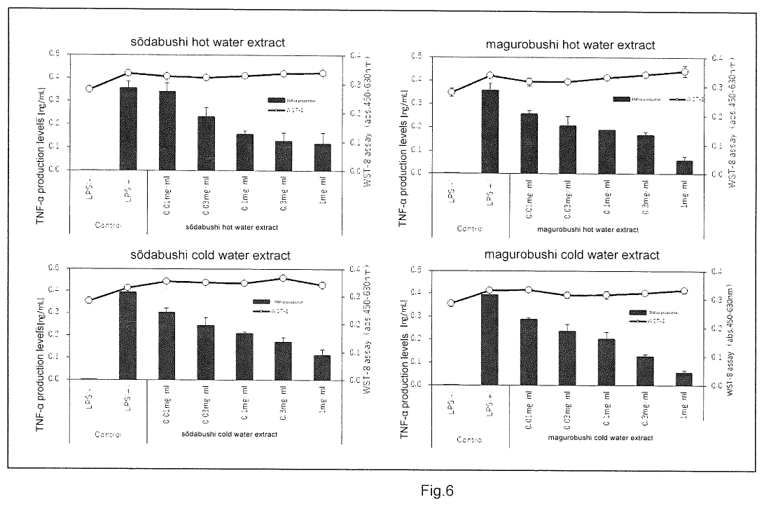

The anti-inflammatory action of hot water extracts and cold water extracts of sōdabushi and magurobushi (0.01 mg, 0.03 mg, 0.1 mg, 0.3 mg, and 1 mg/mL) against LPS stimulation was evaluated (see FIG. 6). From the fact that TNF-α production levels decreased in a concentration-dependent manner regardless of the extraction method used on the sōdabushi or magurobushi, it was revealed that there was anti-inflammatory action. In the WST-8 assay, no reduction in absorption values in the test sections for the various extracts compared to the control test section (LPS±) were observed, from which it could be confirmed that anti-inflammatory action was exhibited without affecting cell viability.

Moreover, when the DHA and EPA content levels of the various extracts were calculated via GCMS analysis, compositions having levels thereof comparable to the extracts were prepared using DHA and EPA reagents, and the anti-inflammatory action thereof was investigated, the anti-inflammatory effects of the various extracts and the compositions containing comparable levels of DHA and EPA were comparable, although the DHA and EPA content levels were not ones at which high anti-inflammatory effects are observed; thus, it was considered highly likely that the substances responsible for the anti-inflammatory effects observed in the various extracts were DHA and EPA.

### [Experiment 7]

As the anti-inflammatory action of the various smoked fish extracts was discovered by the in vitro experiments using MG6 cells from mouse brain microglia, animal experiments involving oral administration to mice were performed as the next step to assay whether effects would be exhibited in vivo as well.

Katsuo-arabushi hot water extract was dissolved with distilled water to a concentration of 11 mg/mL, water bottles were filled therewith and installed on breeding cages, and the animals were allowed to drink freely for four days. Next, the mice were subjected to restraint stress (2 hours), after which the gene expression of inflammatory cytokines in the brain (IL-1β and TNF-α) and blood corticosterone levels were measured. Furthermore, microglial activation in the hypothalamus was observed using immunohistological methods.

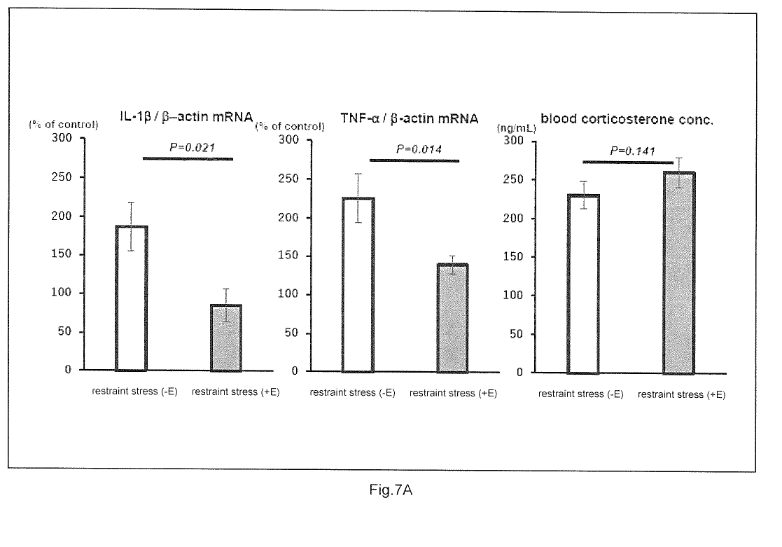

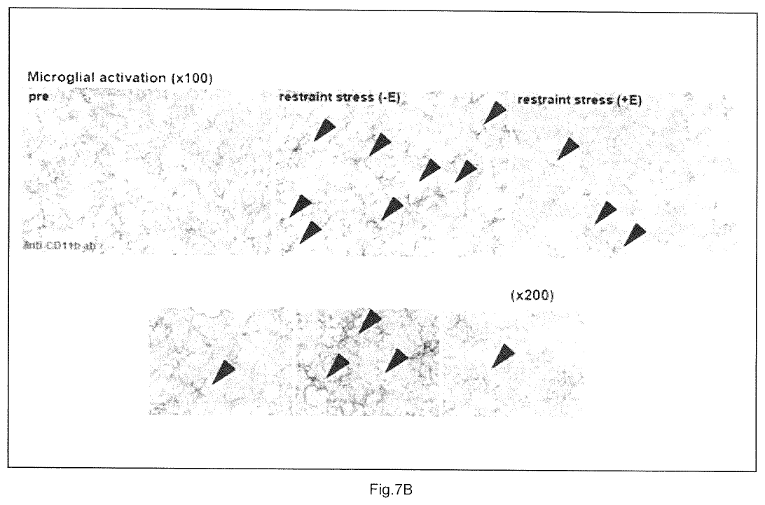

As a result, significant reductions in IL-1β and TNF-α in the brain were observed in the group receiving katsuo-arabushi hot water extract (E) (see FIG. 7A). In addition, microglial activation (the parts marked with black triangles) observed under restraint stress conditions was also significantly inhibited in the group receiving katsuo-arabushi hot water extract (E), and the same conditions as before the restraint stress load could be observed (see FIG. 7B).

From these results, the in vivo experiments also revealed that katsuo-arabushi hot water extract exhibits anti-inflammatory action (inhibition of inflammatory cytokine production and inhibition of microglial activation in the brain).

### [Experiment 8]

The anti-inflammatory action of katsuo-arabushi hot water extract in mice in which inflammation had been induced with LPS was investigated.

Katsuo-arabushi hot water extract was dissolved with water to a concentration of 10 mg/mL, water bottles were filled therewith and installed on breeding cages, and the animals were allowed to drink freely for three days. Next, LPS (10 mg/kg) was injected intraperitoneally, and, four hours later, hepatic gene expression of inflammatory cytokines (TNF-α, IL-1β, and IL-6) and blood inflammatory cytokines (TNF-α and IL-1β) were measured.

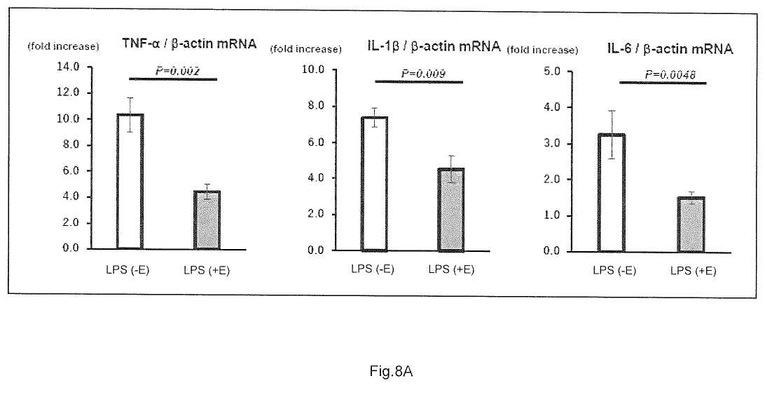

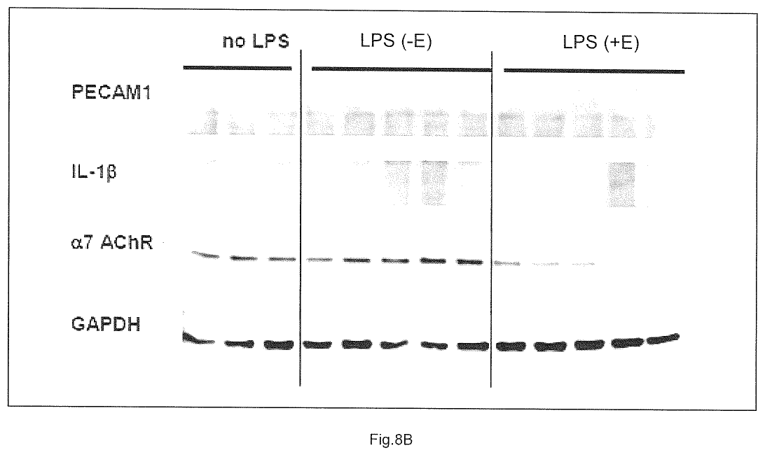

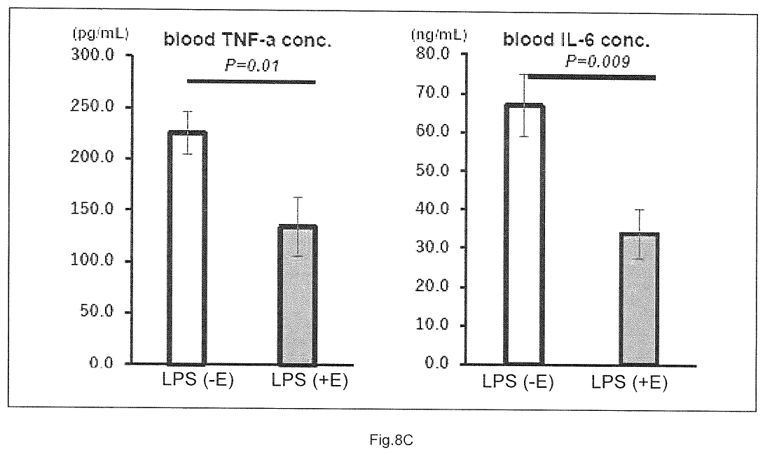

As a result, there was a significant reduction in TNF-α, IL-1β, and IL-6 in the liver in the group receiving katsuo-arabushi hot water extract (E) (see FIG. 8A). At this time, α7 nicotinic receptor (α7AChR) protein expression in the liver decreased, suggesting inhibitory effects by katsuo-arabushi hot water extract (E) on inflammation response (see FIG. 8B). In addition, there was a significant reduction in TNF-α and IL-6 in the blood in the group receiving katsuo-arabushi hot water extract (E) (see FIG. 8C).

From these results, the in vivo experiments also revealed that katsuo-arabushi hot water extract exhibits anti-inflammatory action (inhibition of inflammatory cytokine production in the liver and blood).

### [Experiment 9]

The anti-inflammatory action of fractions obtained by separating and purifying katsuo-arabushi hot water extract via gel-filtration chromatography was assayed using MG6 cells.

First, 1851.6 mg katsuo-arabushi hot water extract (Lot: 200729) was re-dissolved [225 mg/1.5 mL] in 12.3 mL of a mobile phase (0.1 M acetic acid), and separated and purified via gel-filtration chromatography. Gel filtration conditions were as follows: an XK 16/70 column (GE HealthCare) was filled with Sephadex G-25 (GE HealthCare, P/N: 17-0033-02, Lot: 10034186) as a gel filtration medium, the sample was eluted with 0.1 M acetic acid at a flow rate of 0.3 mL/min., and absorbance (214 nm) was measured using a UV detector. The separated eluate was fractionated using a fraction collector for 10 minutes per fraction (3 mL/fraction). Next, each obtained fraction was vacuum-dried and re-dissolved in ultrapure water, fractions with a recovered solids weight of 5 mg or more were re-dissolved in 100 mg/mL ultrapure water, and fractions having a recovered solids weight or less of less than 5 mg were uniformly re-dissolved in 50 μL ultrapure water, after which filtration and sterilization were performed using a 0.2 μm membrane filter. The anti-inflammatory action of each fraction thus prepared was evaluated using MG6 cells.

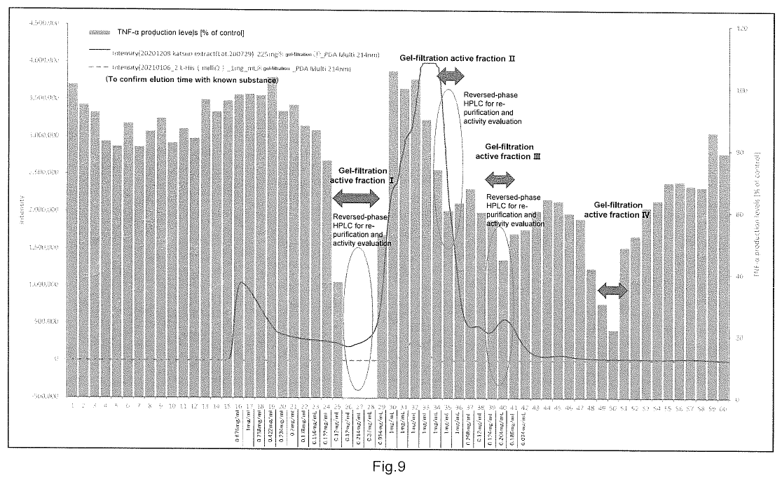

As a result, extremely high anti-inflammatory activity was observed in fractions 26-28 (gel-filtration active fraction I) shown in FIG. 9, and anti-inflammatory activity was also observed in fractions 34-36 (gel-filtration active fraction II), fractions 39-41 (gel-filtration active fraction III), and fractions 48-51 (gel-filtration active fraction IV). However, as gel-filtration active fraction IV had almost no absorption value and difficulty in detection was expected, it was decided to further separate and purify other anti-inflammatory active fractions and attempt to isolate and identify the active ingredients thereof.

First, a gel-filtration active fraction I obtained by separating and purifying katsuo-arabushi hot water extract via gel-filtration chromatography was further separated and purified using reversed-phase HPLC, and the anti-inflammatory action of the obtained fractions were assayed using MG6 cells.

Separation conditions were as follows: using, as a reversed-phase fractionation column, an Inertsil ODS-3, 5 μm, 10×250 mm (GL Science, C/N 5020-06812, S/N 0BI41240), mobile phase A: 0.1% TFA, mobile phase B: 80% acetonitrile—0.1% TFA, a linear concentration gradient of acetonitrile was applied, the sample was eluted at a flow rate of 3 mL/min., and absorbance (214 nm) was measured using a UV detector. The separated eluate was fractionated for 1 minute per fraction (3 mL/fraction). Next, each obtained fraction was vacuum-dried and re-dissolved in ultrapure water, fractions with a recovered solids weight of 5 mg or more were re-dissolved in 100 mg/ml ultrapure water, and fractions having a recovered solids weight of less than 5 mg were uniformly re-dissolved in 50 μL ultrapure water, after which filtration and sterilization were performed using a 0.2 μm membrane filter. The anti-inflammatory action of each fraction thus prepared was evaluated using MG6 cells.

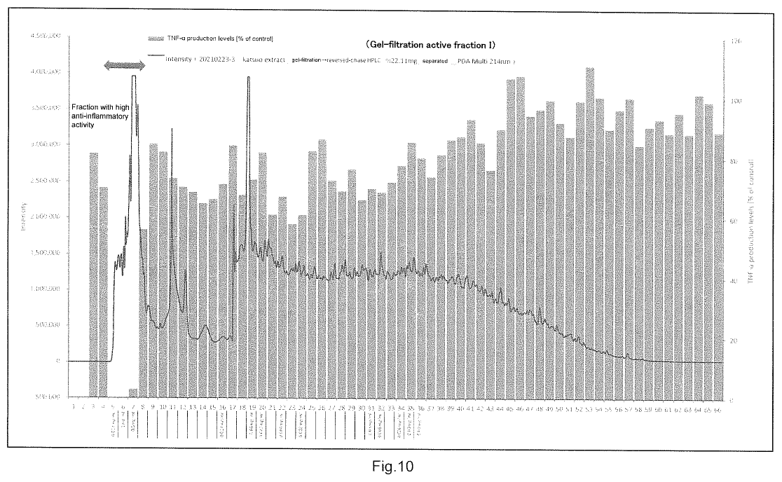

As a result, extremely high anti-inflammatory activity was observed in fractions 5-7 shown in FIG. 10. Estimation of compounds based on precise masses obtained via LCMS analysis suggested the possibilities that fraction 5 contained urea, formate, and 5-hydroxyorotic acid; fraction 6 contained 5-methylcytidine, 2-methylcytidine, and benserazide; and fraction 7 contained lysine-lysine (Lys-Lys), lysine anhydride, lysine-histidine (Lys-His) or (histidine-lysine [His-Lys]), and cadralazine.

Next, a gel-filtration active fraction II obtained by separating and purifying katsuo-arabushi hot water extract via gel-filtration chromatography was further separated and purified using reversed-phase HPLC, and the anti-inflammatory action of the obtained fractions were assayed using MG6 cells.

Separation conditions were as follows: using, as a reversed-phase fractionation column, an Inertsil ODS-3, 5 μm, 10×250 mm (GL Science, C/N 5020-06812, S/N 0BI41240), mobile phase A: 0.1% TFA, mobile phase B: 80% acetonitrile-0.1% TFA, a linear concentration gradient of acetonitrile was applied, the sample was eluted at a flow rate of 3 mL/min., and absorbance (214 nm) was measured using a UV detector. The separated eluate was fractionated for 1 minute per fraction (3 mL/fraction). Next, each obtained fraction was vacuum-dried and re-dissolved in ultrapure water, fractions with a recovered solids weight of 5 mg or more were re-dissolved in 100 mg/ml ultrapure water, and fractions having a recovered solids weight of less than 5 mg were uniformly re-dissolved in 50 μL ultrapure water, after which filtration and sterilization were performed using a 0.2 μm membrane filter. The anti-inflammatory action of each fraction thus prepared was evaluated using MG6 cells.

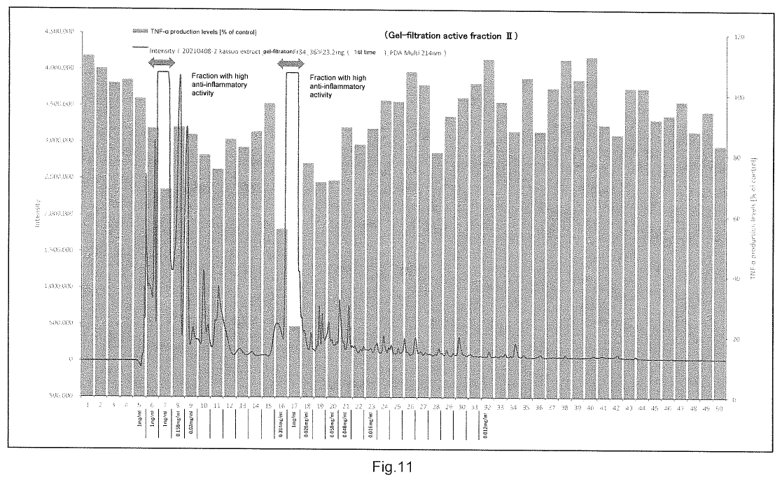

As a result, anti-inflammatory activity was observed in fractions 7 and 17 shown in FIG. 11. Estimation of compounds based on precise masses obtained by LCMS analysis suggested the possibility that fraction 7 contained creatine, creatinine, glycolic acid, and lactic acid; and fraction 17 contained inosinic acid, AMP, succinic acid, ribose 5-phosphate, and hypoxanthine.

To attempt to isolate and identify substances exhibiting anti-inflammatory action, the anti-inflammatory action of the four components (creatine, creatinine, glycolic acid, and lactic acid) detected in quantity from fraction 7 was evaluated for each component in isolation.

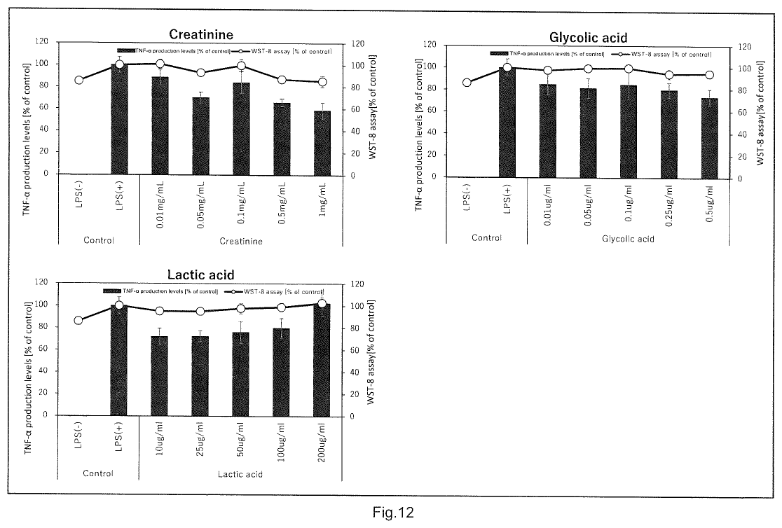

Of the four components (creatine, creatinine, glycolic acid, and lactic acid) contained in fraction 7, three components (creatinine, glycolic acid, and lactic acid) showed anti-inflammatory activity (see FIG. 12). Of these, lactic acid has already been reported to have anti-inflammatory activity (Liang et al., L-lactate inhibits lipopolysaccharide-induced inflammation of microglia in the hippocampus, International Journal of Neuroscience, 2022 Jul. 26; 1-8), but the other two components (creatinine and glycolic acid) were novel anti-inflammatory components.

Next, the anti-inflammatory action of the five components (inosinic acid, AMP, succinic acid, ribose 5-phosphate, and hypoxanthine) detected in quantities from fraction 17 was evaluated for each component in isolation.

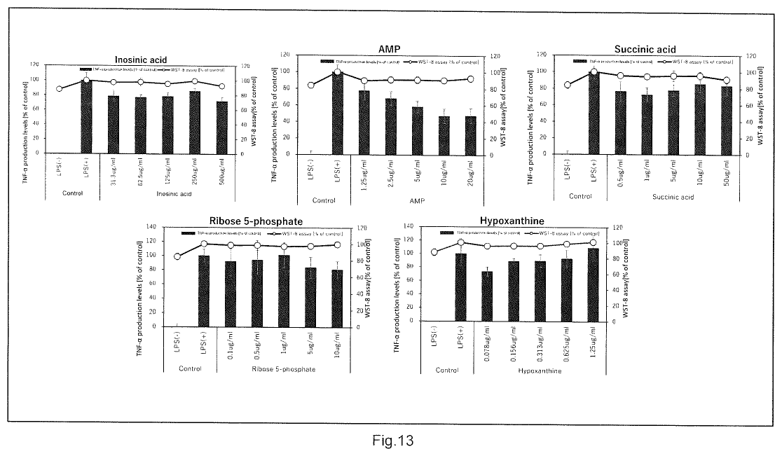

As a result, anti-inflammatory activity was observed in each of inosinic acid, AMP, succinic acid, ribose 5-phosphate, and hypoxanthine (see FIG. 13).

In other words, the possibility that the seven components creatinine, glycolic acid, inosinic acid, AMP, succinic acid, ribose 5-phosphate, and hypoxanthine might be novel anti-inflammatory components contained in katsuobushi hot water extract was suggested.

Finally, a gel-filtration active fraction III obtained by separating and purifying katsuo-arabushi hot water extract via gel-filtration chromatography was further separated and purified using reversed-phase HPLC, and the anti-inflammatory action of the obtained fractions were assayed using MG6 cells.

Separation conditions were as follows: using, as a reversed-phase fractionation column, an Inertsil ODS-3, 5 μm, 10×250 mm (GL Science, C/N 5020-06812, S/N 0BI41240), mobile phase A: 0.1% TFA, mobile phase B: 80% acetonitrile-0.1% TFA, a linear concentration gradient of acetonitrile was applied, the sample was eluted at a flow rate of 3 mL/min., and absorbance (214 nm) was measured using a UV detector. The separated eluate was fractionated for 1 minute per fraction (3 mL/fraction). Next, each obtained fraction was vacuum-dried and re-dissolved in ultrapure water, fractions with a recovered solids weight of 5 mg or more were re-dissolved in 100 mg/ml ultrapure water, and fractions having a recovered solids weight or less of less than 5 mg were uniformly re-dissolved in 50 μL ultrapure water, after which filtration and sterilization were performed using a 0.2 μm membrane filter. The anti-inflammatory action of each fraction thus prepared was evaluated using MG6 cells.

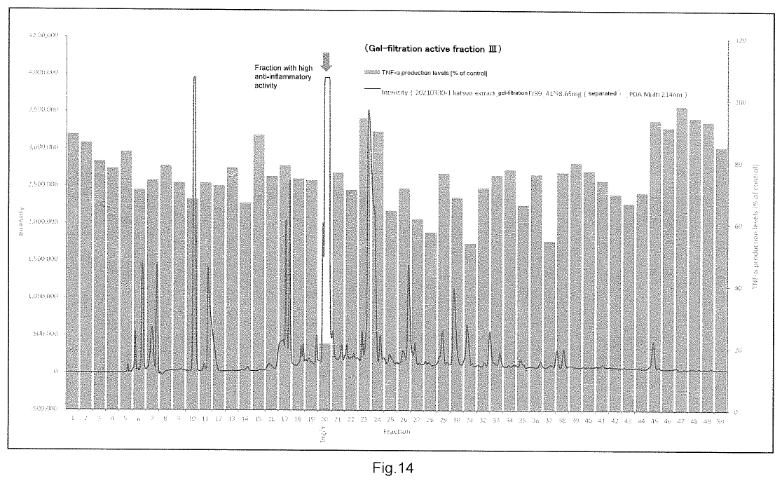

As a result, extremely high anti-inflammatory activity was observed in fraction 20 shown in FIG. 14. Estimation of compounds based on precise masses obtained by LCMS analysis suggested the possibility that fraction 20 contained inosine and arabinosylhypoxanthine.

### [Experiment 10]

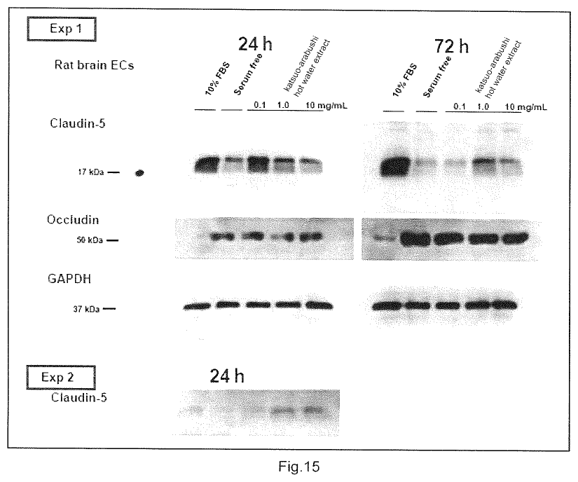

To investigate the effects of katsuo-arabushi hot water extract on BBB function, assays were performed via in vitro experimentation, using protein expression of the tight-junction-associated proteins claudin 5 and occludin in rat brain capillary endothelial cells as a benchmark. Claudin-5 and occludin, which form tight junctions, the barriers to vascular endothelial cell gaps, are commonly used as markers for evaluating BBB function (see Cerebral Circulation Metabolism 24:111-115, 2013).
Protein expression of tight-junction-associated molecules in rat brain capillary endothelial cells (RBECs, primary cells) (see FIG. 15)

Rat brain capillary endothelial cells (RBECs, primary cells), medium, and the like were purchased from PharmaCo-Cell, and the effects of katsuo-arabushi hot water extract on protein expression of tight-junction-associated molecules (claudin-5 and occludin) were assayed.

First, a 48-well plate was seeded with the RBECs (2×10⁵ cells/well·440 μL), and a culture was started in a CO₂ incubator (37° C., CO₂ level 5%). Next, after 72 hours, the medium in each well was replaced with an evaluation medium prepared by adding the various extracts, and, 24 hours and 72 hours after that, RNA and protein were recovered from the cultured cells a NucleoSpin RNA/Protein kit (Takara Bio) according to the protocol of the kit. The extracted protein was evaluated via western blot, and DNA obtained by reverse transcription of the extracted RNA was evaluated via real-time PCR.

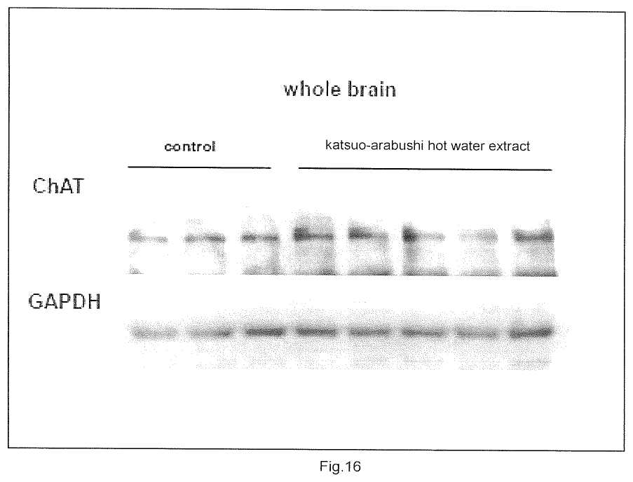

As a result, the protein expression of claudin-5 was elevated after 24 and 72 hours compared to a serum-free medium test section, and occludin expression was also elevated, especially after 24 hours, as if interacting with the claudin-5 (EXP 1). A similar assay of RBECs passaged another two times (total passages: 5) confirmed that protein expression of claudin-5 was elevated compared to a serum-free medium test section [EXP 2].
Acetylcholine synthase in mouse brains (see FIG. 16)

Mice received 10 mg/ml oral katsuo-arabushi hot water extract, after which the protein expression level of acetylcholine synthase (ChAT) was assayed from prepared whole-brain samples (control group: 3 mice; katsuo-arabushi hot water extract-receiving group: 5 mice).

As a result, elevated ChAT protein expression was observed in whole-brain extract preparations, suggesting elevated acetylcholine production in cerebral neurons.

From these results, katsuo-arabushi hot water extract was found to elevate protein expression of tight-junction-associated molecules (claudin-5 and occludin) in brain capillary endothelial cells in in vivo experiments.

### [Experiment 11]

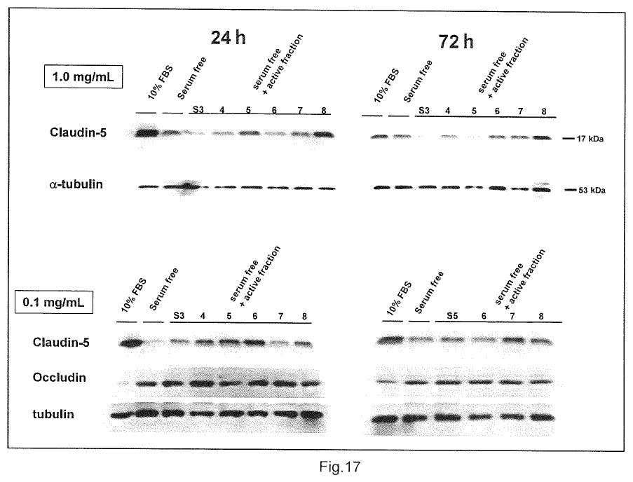

The effects of highly-active anti-inflammatory fractions obtained by sequentially separating and purifying katsuo-arabushi hot water extract via gel-filtration chromatography and reversed-phase HPLC upon protein expression of tight-junction-associated molecules (claudin-5 and occludin) in rat brain capillary endothelial cells (RBECs, primary cells) were assayed (see FIG. 17).

Rat brain capillary endothelial cells (RBECs, primary cells), medium, and the like were purchased from PharmaCo-Cell, and the effects of katsuo-arabushi hot water extract on protein expression of tight-junction-associated molecules (claudin-5 and occludin) were assayed. First, a 48-well plate was seeded with the RBECs (2×10⁵ cells/well·440 μL), and a culture was started in a CO₂ incubator (37° C., CO₂ level 5%). Next, after 72 hours, the medium in each well was replaced with an evaluation medium prepared by adding the various extracts, and, 24 hours and 72 hours later, RNA and protein were recovered from the cultured cells a NucleoSpin RNA/Protein kit (Takara Bio) according to the protocol of the kit. The extracted protein was evaluated via western blot, and DNA obtained by reverse transcription of the extracted RNA was evaluated via real-time PCR (real-time PCR data not shown here).

As a result, when the added active fraction concentration was 0.1 mg/ml, claudin-5 protein expression was elevated compared to a serum-free medium test section after 24 hours in samples 4, 5, and 6 (corresponding to fraction 20 in FIG. 14 and fractions 5 and 6 in FIG. 10, respectively) and after 72 hours in samples 7 and 8 (corresponding to fraction 7 in FIG. 10 and fraction 7 in FIG. 11, respectively). When the added active fraction concentration was 1.0 mg/ml, claudin-5 protein expression was elevated compared to a serum-free medium test section after 24 hours in sample 5 (corresponding to fraction 5 in FIG. 10) and sample 8 (corresponding to fraction 7 in FIG. 11), and after 72 hours in sample 8 (corresponding to fraction 7 in FIG. 11).

From these results, it was revealed that the highly-active anti-inflammatory fractions obtained by sequentially separating and purifying katsuo-arabushi hot water extract via gel-filtration chromatography and reversed-phase HPLC elevate protein expression of tight-junction-associated molecules (claudin-5 and occludin) in rat brain capillary endothelial cells (RBECs, primary cells).

### [Experiment 12]

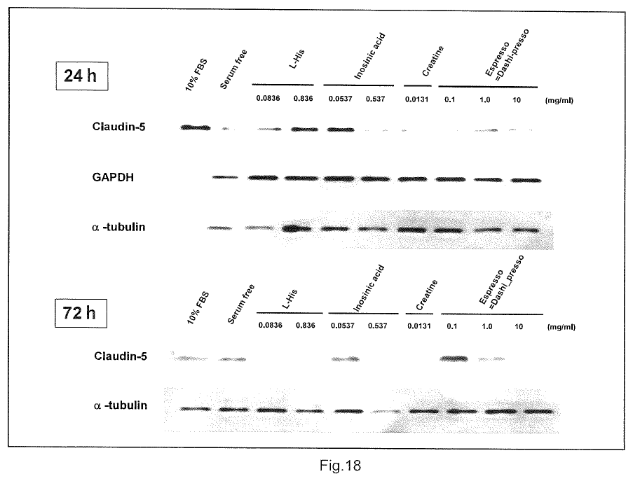

The effects of compounds (inosinic acid and histidine) that were estimated, on the basis of precise atomic numbers obtained via LCMS analysis, to be components of a highly-active anti-inflammatory fraction by sequentially separating and purifying katsuo-arabushi hot water extract via gel-filtration chromatography and reverse-phase HPLC, as well as the effects of Dashi-presso (boxed katsuo-dashi produced by Maruhachi Muramatsu, inc.), upon protein expression of tight-junction-associated molecules (claudin-5 and occludin) in rat brain capillary endothelial cells (RBECs, primary cells) were assayed (see FIG. 18).

Rat brain capillary endothelial cells (RBECs, primary cells), medium, and the like were purchased from PharmaCo-Cell, and the effects of katsuo-arabushi hot water extract on protein expression of tight-junction-associated molecules (claudin-5 and occludin) were assayed. First, a 48-well plate was seeded with the RBECs (2×10⁵ cells/well·440 μL), and a culture was started in a CO₂ incubator (37° C., CO₂ level 5%). Next, after 72 hours, the medium in each well was replaced with an evaluation medium prepared by adding various components and extracts, and 24 hours and 72 hours later, RNA and protein were recovered from the cultured cells a NucleoSpin RNA/Protein kit (Takara Bio) according to the protocol of the kit. The extracted protein was evaluated via western blot, and DNA obtained by reverse transcription of the extracted RNA was evaluated via real-time PCR (real-time PCR data not shown here).

As a result, protein expression claudin-5 in the inosinic acid (added concentration: 0.0537 mg/mL) and histidine (added concentration: 0.836 mg/ml) was elevated compared to a serum-free medium test section after 24 hours. In the case of Dashi-presso (added concentration: 0.1 mg/mL), claudin-5 protein expression was elevated compared to a serum-free medium test section after 72 hours.

From these results, it was revealed that the components (inosinic acid and histidine) expected to be contained in highly-active anti-inflammatory fractions obtained by sequentially separating and purifying katsuo-arabushi hot water extract via gel-filtration chromatography and reversed-phase HPLC elevate protein expression of tight-junction-associated molecules (claudin-5) in rat brain capillary endothelial cells (RBECs, primary cells).

### [Experiment 13]

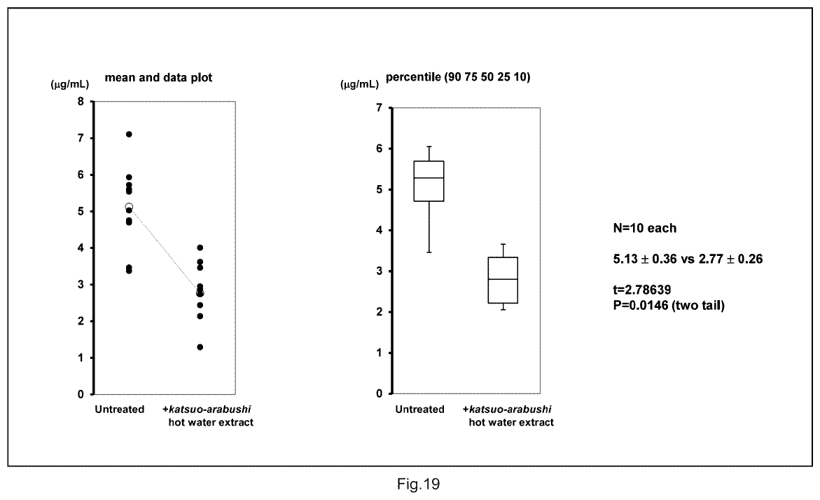

Cerebrovascular permeability after receiving oral katsuo-arabushi hot water extract before and after physical injury to the blood-brain barrier was evaluated using Evans blue (EB) (see FIG. 19).

Specifically, mice were given 10 mg/ml oral katsuo-arabushi hot water extract for 3 days, and on day 4, while performing oral administration, a chilled metal rod (diameter: 3 mm) was placed in contact with the right parietal bone of the skull for 5 seconds to directly damage the blood-brain barrier. Twenty-four hours after infliction of cryoinjury, 3% EB was administered, and, after 3-4 hours, a 3-mm-thick section of the right cerebral hemisphere was taken and immersed in 800 μL of 50° C. formaldehyde for 3 days. Absorbance was then measured at 634 nm.

As a result, the quantity of EB leaking into the brain was clearly less in the group receiving katsuo-arabushi hot water extract than in the non-receiving group, from which fact it was revealed that katsuo-arabushi hot water extract is highly effective in maintaining and improving blood-brain barrier function.

### [Experiment 14]

Effects on cardiac acetylcholine production capacity and hemodynamic changes were investigated.

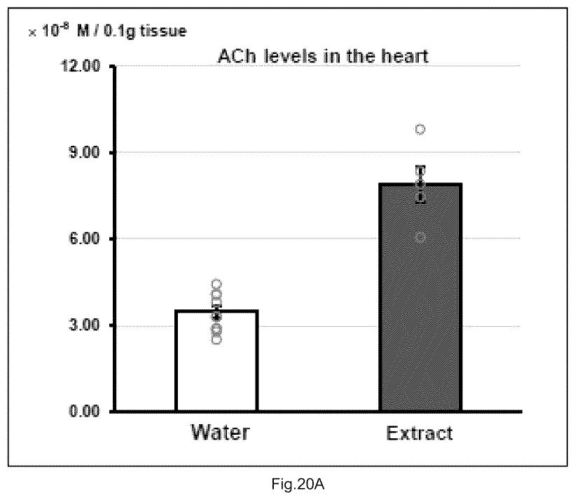

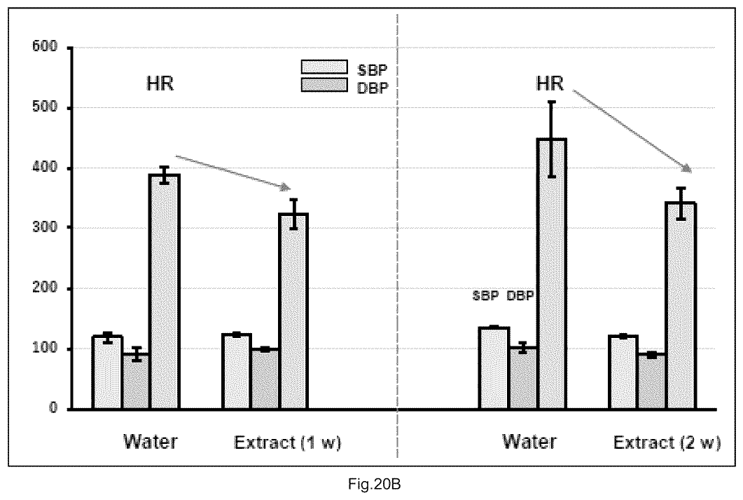

Comparison of a group of mice receiving 10 mg/mL katsuo-arabushi hot water extract for two weeks with a group receiving water revealed a clear increase in cardiac acetylcholine levels (see FIG. 20A), as well as elevated expression of acetylcholine synthase (ChAT) at the protein level in the heart as well as the brain (see FIG. 16).

The effects of katsuo-arabushi hot water extract on blood pressure and heart rate in mice receiving oral katsuo-arabushi hot water extract for one or two weeks were investigated. There was clearly a marked decrease in heart rate (HR), more so in the two-week group than the one-week group, compared to the water-receiving group, as well as slight downward trends in systolic blood pressure (SBP) and diastolic blood pressure (DBP) in the two-week group. In other words, it was suggested that katsuo-arabushi hot water extract elevates parasympathetic nervous system activity and cardiac acetylcholine production capacity in mice (see FIG. 20B).

### [Experiment 15]

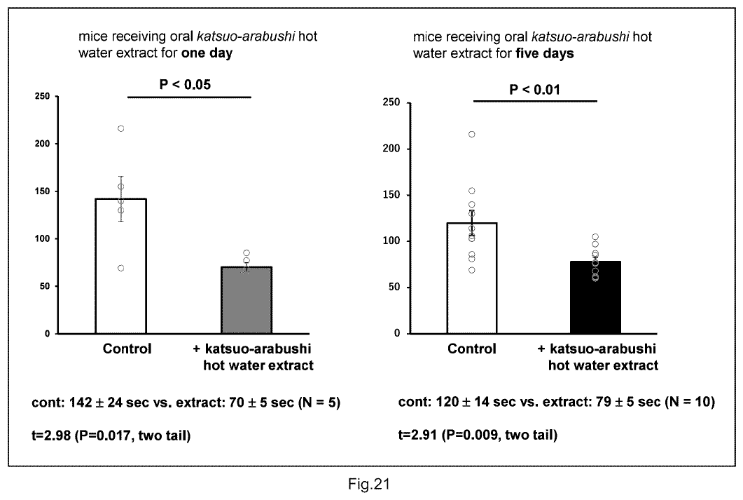

A forced swim test (FST) was conducted using mice receiving 10 mg/ml of oral katsuo-arabushi hot water extract to investigate inhibitory effects upon depressive-like behaviors (see FIG. 21).

Specifically, mice receiving oral katsuo-arabushi hot water extract for one or five days were forced to swim in a water-filled aquarium and observed for 10 minutes, and the length of time the mice remained in an immobile state in the last four minutes was measured. Long immobility time was considered to indicate a strong depressive state, and short immobility time was considered to indicate antidepressant action.

As a result, it was revealed that katsuo-arabushi hot water extract played a role in antidepressant action in two experiments having different oral administration periods.

### [Experiment 16]

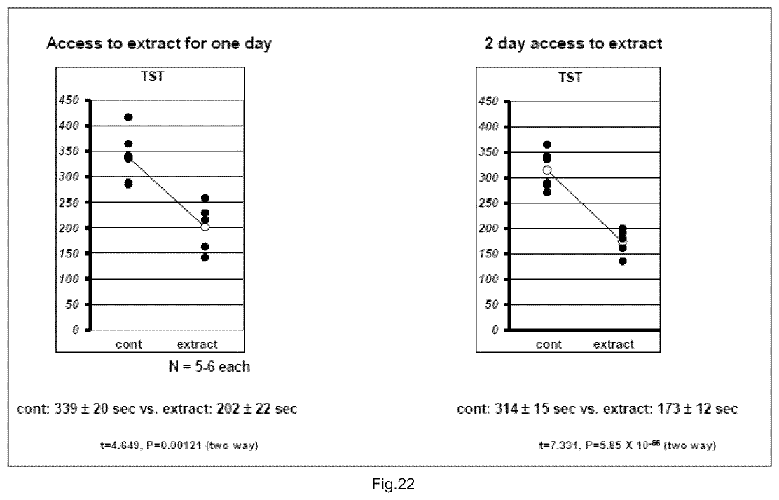

A tail suspension test (TST) was conducted using mice receiving 10 mg/ml of oral katsuo-arabushi hot water extract to investigate inhibitory effects on depressive-like behaviors (see FIG. 22).

Specifically, mice receiving oral katsuo-arabushi hot water extract for one or two days were suspended in place by their tails and observed for 10 minutes, and the length of time the mice remained in an immobile state was measured. Long immobility time was considered to indicate a strong depressive state, and short immobility time was considered to indicate antidepressant action.

As a result, it was revealed that katsuo-arabushi hot water extract played a role in antidepressant action in two experiments having different oral administration periods.

### [Experiment 17]

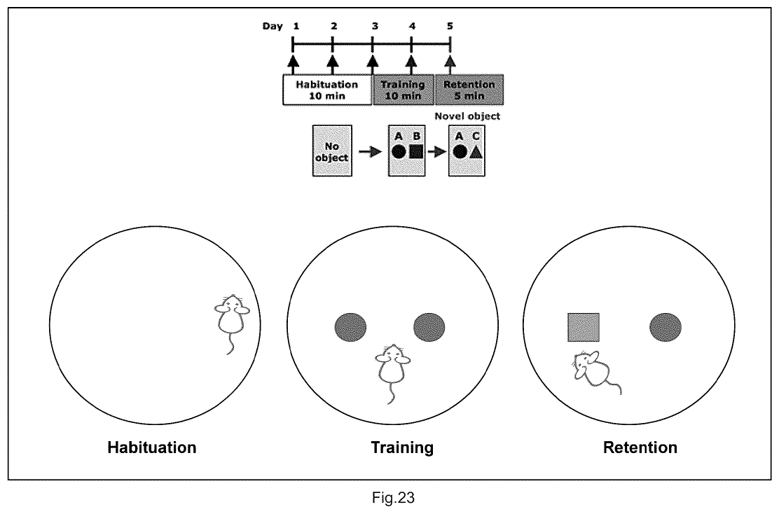

A novel object recognition test, which is a method for evaluating visual cognitive memory, taking advantage of the characteristic preference of mice for novelty was conducted as shown in FIG. 23, and effects upon recognition and memory of novel objects were compared between two groups, one receiving katsuo-arabushi hot water extract and one not receiving extract.

First, a mouse was placed in an experimental device (a cylindrical cylinder having a diameter of apparatus 50 cm) in which no object had been placed and allowed to acclimate to the environment for 10 minutes (habituation), after which two identical objects were placed in the experimental device and the mouse was allowed to explore freely for 10 minutes (training). One object was then replaced with a new object, and the mouse was allowed to explore freely for 10 minutes (retention). The movements of the mouse were recorded from above by an installed camera. The lengths of time each of the two objects was explored and total exploration time in the training and retention phases were measured. Exploration preference was calculated as the percentage (%) of exploration time for one of the objects relative to total exploration time in the training phase and as the percentage (%) of exploration time for the novel object relative to total exploration time in the retention phase, and the latter was used as a benchmark of visual cognitive memory.

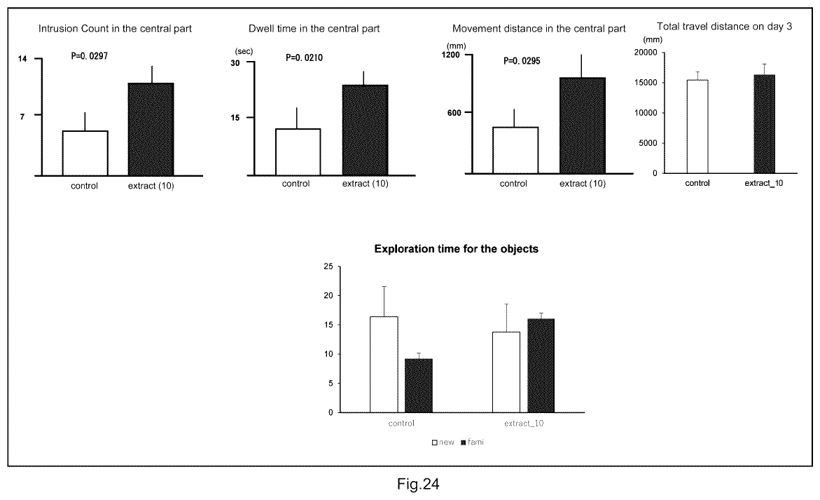

As a result, substantially no difference in total travel distance on day 3 was observed between the groups receiving and not receiving 10 mg/ml katsuo-arabushi hot water extract, but significant increases in the time the mice entered an area designated as the central part of the experimental device, residence time in said area, and movement distance in the central part were observed in the group receiving katsuo-arabushi hot water extract. Moreover, time until engaging in exploratory behavior (exploration latency) was longer for the novel object than the known object in the group not receiving katsuo-arabushi hot water extract (control; water), whereas there was substantially no difference in exploration latency between the novel object and the known object in the group receiving katsuo-arabushi hot water extract (see FIG. 24).

From these findings, it was revealed that ingestion of katsuo-arabushi hot water extract resulted in a tendency to explore both novel and known objects without distinction, perhaps because of reduced fear. In other words, it was believed that katsuo-arabushi hot water extract might have the effect of alleviating fear of novel objects.

1. A functional food for use in improving blood-brain barrier properties, the functional food being characterized by comprising bonito-derived extract.

2. (canceled)

3. (canceled)

4. (canceled)

5. (canceled)

6. (canceled)

7. (canceled)

8. (canceled)

9. The functional food according to claim 1, wherein the active ingredients in the effect of improving blood-brain barrier properties are histidine and inosinic acid.

10. (canceled)

11. The functional food according to claim 1, wherein the concentration of the bonito-derived extract is 0.1 mg/mL.

12. The functional food according to claim 9, wherein the concentration of the bonito-derived extract is 0.1 mg/mL, or the concentration of histidine is 0.836 mg/mL and the concentration of inosinic acid is 0.0537 mg/mL.

13. (canceled)
